(19) United States
(12) Patent Application Publication
(10) Pub. No.: US 2009/0325921 A1
(43) Pub. Date: Dec. 31, 2009

(54) METHOD OF IMPROVING THE OVERALL HEALTH OF A PATIENT WITH USE OF A TRANSDERMAL DHEA CREAM AND/OR DHEA SULFATE CREAM

(76) Inventor: John R. Woodward, Dallas, TX (US)

Correspondence Address: Michael A. O'Neil. P.C., 8150 North Central Expressway, Suite 1150, DALLAS, TX, 75206, US

(21) Appl. No.: 12/490,076

(22) Filed: Jun. 23, 2009

Related U.S. Application Data
(60) Provisional application No. 61/075,602, filed on Jun. 25, 2008.

(51) Int. Cl. A61K 31/56

(52) U.S. Cl. 514/178

(57) ABSTRACT

A method is described herein for improving the overall health of a patient by providing a topical DHEA cream and applying the topical DHEA cream to at least one part of a body. In another embodiment, the topical DHEA cream is mixed with a moisturizing lotion and the resulting DHEA/lotion mixture is applied to at least one part of a body. In yet another embodiment, the topical DHEA cream is mixed with a sulfate lotion and the resulting DHEA/sulfate lotion combination is applied to at least one part of a body.

This application claims the benefit of U.S. Provisional Application Ser. No. 61/075,602 filed on Jun. 25, 2008, the contents of which are hereby incorporated by reference herein.

### BRIEF DESCRIPTION OF THE FIGURES

For a more complete understanding of the present invention, including its features and advantages, reference is now made to the detailed description of the invention taken in conjunction with the accompanying drawing in which:

FIG. 1 is a diagram utilized to explain an embodiment of the present invention.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

For many years dehydroepiandrosterone (DHEA) has been studied and found to have positive effects on patients. DHEA is the most common steroid hormone in the body. It is produced in the adrenal glands and within the skin. DHEA is metabolized from pregnenolone, the body's “master hormone,” which itself is metabolized from cholesterol. DHEA production has also been found to decrease as humans get older and seems to peak when a person is about 25 years old. DHEA replacement therapy normally consists of patients taking oral supplements but the therapy is still evolving.

An embodiment of the present invention utilizes a topical DHEA cream consisting about 5% DHEA and is applied directly on the skin. Application directly on the skin helps replenish the DHEA normally produced within the skin in patients over 25. Consequently, direct skin application has been found to improve the overall health of a patient. In addition, positive effects have been found if the cream is applied to thin vascular skin such as between the inner arm and the side of the arm or flank.

Another embodiment utilizes the topical DHEA cream with a body moisturizing lotion. In this embodiment, Twist25® topical DHEA cream and St. Ives® with vitamin E lotion is used, however, other similar products can also be used. This embodiment contemplates mixing an equal part of the lotion and the topical DHEA cream together and applying the topical DHEA/lotion mixture to the body of a patient.

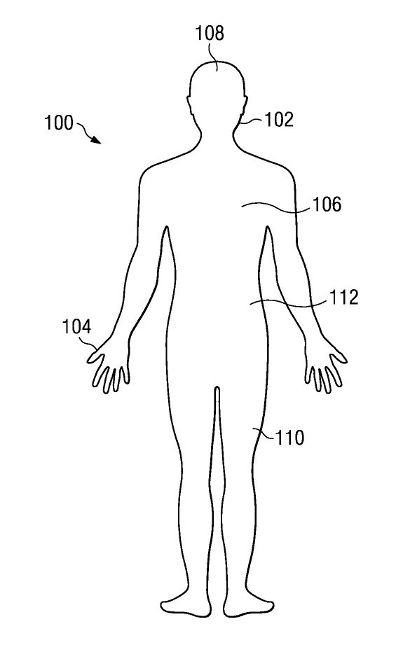

FIG. 1 illustrates an example patient 100. In this embodiment the DHEA/lotion is applied topically to skin on the face 102, hands 104 and the chest 106 of the patient 100. This embodiment contemplates two applications per day in each of those areas. Applications of this type have shown to positively effect the mood and well-being of patients. In addition, these types of applications have also shown to decrease pro-inflammatory cytokine secretion production stimulated by ACTH and stress. Such, the applications have been shown to improve the muscle to fat ratio of patients.

DHEA/lotion can also be applied to the nipples and clitoris of a female patient and the head of a male's penis. This application has been shown to improve libido and sexual response of the patients. One reason that this type of application helps is because the combined DHEA/lotion mixture has been shown to have a strong affinity for the Sex Hormone Binding Globulin (SHBG).

Additionally, the topical DHEA cream can also be used with a body and massage lotion that contains sulfate, such as Lasting Touch™ Deep Tissue Therapy and Massage lotion, for additional benefits. In this embodiment the topical DHEA cream is also combined with an equal portion of the sulfate lotion (s-lotion). Then, the combined DHEA/s-lotion mixture is applied to the scalp 108, knees 110 and the waist 112 of the patient 100. These types of applications have a strong affinity for serum albumin and positively effect cognition, depression and mobility. In addition, the applications affect global self-rated health for men and especially women. Moreover, the applications lower blood pressure and improve cardiovascular health in men. Further, the applications are antagonistic to cortisol and thus decrease abdominal fat. The applications also improve the skin and joints and increase hair growth.

Although this invention has been described with reference to an illustrative embodiment, this description is not intended to limit the scope of the invention. Various modifications and combinations of the illustrative embodiments as well as other embodiments of the invention will be apparent to persons skilled in the art upon reference to the description. It is therefore intended that the claims accomplish any such modifications or embodiments.

We claim:

1. A method of improving the overall health of a patient, the method comprises:
providing a topical DHEA cream;
applying the topical DHEA cream to at least one part of a body.

2. The method of claim 1 wherein the topical DHEA cream is applied to a face of the body.

3. The method of claim 1 wherein the topical DHEA cream is applied to hands of the body.

4. The method of claim 1 wherein the topical DHEA cream is applied to a chest of the body.

5. The method of claim 1 wherein the topical DHEA cream is applied to knees of the body.

6. The method of claim 1 wherein the topical DHEA cream is applied to a waist of the body.

7. The method of claim 1 wherein the topical DHEA cream is applied to a scalp of the body.

8. A method of improving the overall health of a patient, the method comprises:
providing a topical DHEA cream;
providing a moisturizing lotion;
mixing portions of the topical DHEA cream and moisturizing lotion together to create a DHEA/lotion mixture; and
applying the DHEA/lotion mixture to at least one part of a body.

9. The method of claim 8 wherein the DHEA/lotion mixture is applied to a face of the body.

10. The method of claim 8 wherein the DHEA/lotion mixture is applied to hands of the body.

11. The method of claim 8 wherein the DHEA/lotion mixture is applied to a chest of the body.

12. The method of claim 8, wherein the DHEA/lotion mixture is applied to at least a nipple and clitoris of the body.

13. The method of claim 8, wherein the DHEA/lotion mixture is applied to a penis of the body.

14. A method of improving the overall health of a patient, the method comprises:
providing a topical DHEA cream;
providing a lotion with sulfate;
mixing portions of the topical DHEA cream and lotion with sulfate together to create a DHEA/sulfate lotion combination; and
applying the DHEA/sulfate lotion mixture to at least one part of the body.

15. The method of claim 14 wherein the DHEA/sulfate lotion combination is applied to knees of the body.

16. The method of claim 14 wherein the DHEA/sulfate lotion combination is applied to a waist of the body.

17. The method of claim 14 wherein the DHEA/sulfate lotion combination is applied to a scalp of the body.
